(19) United States
(12) Patent Application Publication
(10) Pub. No.: US 2024/0374916 A1
(43) Pub. Date: Nov. 14, 2024

(54) LIGHT THERAPY SYSTEM AND METHOD

(71) Applicant: KORRUS, INC., Los Angeles, CA (US)

(72) Inventors: Benjamin Harrison, Los Angeles, CA (US); Paul Kenneth Pickard, Los Angeles, CA (US); Levent Sahin, Los Angeles, CA (US)

(21) Appl. No.: 18/780,219

(22) Filed: Jul. 22, 2024

Related U.S. Application Data
(63) Continuation of application No. PCT/US23/11238, filed on Jan. 20, 2023.
(60) Provisional application No. 63/301,414, filed on Jan. 20, 2022.
(60) Provisional application No. 63/403,068, filed on Sep. 1, 2022.

(51) Int. Cl. A61N 5/06; A61M 21/00

(52) U.S. Cl. CPC A61N 5/0618; A61M 21/00; A61N 5/0622; A61M 2021/0011; A61N 2005/0626; A61N 2005/0642; A61N 2005/0654; A61N 2005/0661; A61N 2005/0663

(57) ABSTRACT

A system for delivering light therapy, the system comprising: (a) a first light source for emitting continuous light; and (b) a second light source for emitting pulsed light having a frequency of between 40 and 200 Hz in integer multiples of 20 Hz.

### CROSS-REFERENCE TO RELATED APPLICATIONS

This application claims benefit to U.S. Provisional Application No. 63/301,414, filed on Jan. 20, 2022, and to U.S. Provisional Application No. 63/403,068, filed on Sep. 1, 2022, each of which is incorporated by reference in its entirety.

### FIELD OF INVENTION

The subject matter herein relates, generally, to light therapy, and, more specifically, to a system and method of using pulsing light to entrain brain waves in a user.

### BACKGROUND

The electrical activity in the brain depends upon the type of activity being done by a person. For example, the brain waves of a person who is reading are very different to those of a person who is relaxing. These brain waves/rhythms are classified in five categories-namely, gamma waves, beta waves, alpha waves, theta wave, and delta waves. Each of these waves provide information about a person's health and state of mind. Of particular interest herein are gamma waves.

A gamma wave is considered to be the fastest brain activity. It is important for cognitive functioning, learning, memory, and information processing. In optimal conditions gamma waves help with attention, focus, binding of senses (smell, sight, and hearing), consciousness, mental processing, and perception. But suppression of these waves can lead to Attention Deficit Hyperactivity Disorder (ADHD), depression, and learning disabilities. Moreover, diminished gamma activity is seen in human and mouse models of Alzheimer's disease (AD). More specifically, the reduction in gamma activity may increase Amyloid Beta (Aβ) in the brain, since gamma activity is thought to be involved in Aβ elimination. Abnormal aggregation of Amyloid Beta (Aβ) in the brain is a hallmark of AD and has been associated with impaired cognitive performance.

Gamma activity can be entrained through external stimuli. Brainwaves have been found to entrain a variety of external stimuli, including sound, visible light, and non-visible light (e.g., infrared). Of particular interest herein is light therapy for entraining brainwaves or brainwave light therapy (BLT), for short. Entrainment is achieved by flickering the stimuli at the desired frequency. For example, one hour of 40 Hz optogenetic stimulation of parvalbumin interneurons in the CA1 region of the hippocampus has been shown to reduce Aβ levels by approximately 50% in 5XFAD mice. A similar reduction was detected in the visual cortex after mice were exposed to a 40 Hz flickering light. Furthermore, induced gamma waves increased microglial responses in 5XFAD, Tau P301S, and CK-p25 animals as well as in healthy mice via elevated cytokine, which is important since microglia provide protective functions that limit Aβ buildup and prevent the onset of AD.

Aside from degradation of gamma waves in the brain, the destruction of circadian rhythms may also contribute to AD. In approximately 45% of Alzheimer's patients, circadian rhythm problems and sleep disruptions have been recorded, where a bidirectional association between sleep and Alzheimer's disease is suggested, with sleep abnormalities serving as either a marker for AD pathology or a mechanism mediating increased risk of the disease. Hippocampal memory has been found to be impaired when the circadian rhythm is disrupted on a long-term basis which is associated with the neuronal loss in the suprachiasmatic nucleus (SCN) of the circadian master clock. The output of SCN can also be impaired by the buildup of Aβ. AD patients also have lower levels and irregular secretion rhythms of melatonin, which not only regulates circadian rhythms but also, protects against oxidative stress, which has been linked to AD development.

Applicant believes that circadian and brainwave interventions are most effective when the intervention is performed over a long time. For example, treatment times in terms of years or even decades are likely to be necessary to produce meaningful results. However, pulsed sound and light for inducing entrainment can be highly perceptible and annoying. Applicant recognizes that a person is unlikely to comply with a BLT that requires staring at an obnoxious flickering light every day for years. Applicant therefore recognizes the importance of seamlessly/invisibly integrating a BLT with a person's behavior/lifestyle/work environment to ensure adequate exposure and effective results. Therefore, to realize acceptable compliance, there is a need for a BLT that is an innocuous part of a person's daily regimen. The present invention fulfills this need, among others.

### SUMMARY

The following presents a simplified summary of the invention in order to provide a basic understanding of some aspects of the invention. This summary is not an extensive overview of the invention. It is not intended to identify key/critical elements of the invention or to delineate the scope of the invention. Its sole purpose is to present some concepts of the invention in a simplified form as a prelude to the more detailed description that is presented later.

Applicant discloses herein an approach for seamlessly/invisibly integrating brainwave light therapy (BLT) with a person's behavior/lifestyle/work environment to ensure adequate exposure and effective results. To this end, Applicant discloses approaches to minimize the intrusiveness of BLT. Applicant also discloses approaches for synergistically combining BLT with other light therapies. Furthermore, Applicant discloses incorporating BLT in devices that are used daily voluntarily, such as displays and lighting, to ensure long term compliance with the BLT. Thus, the present invention provides for a non-obtrusive light therapy that can entrain desired brainwaves, while moderating other physiological conditions (e.g., circadian cycle, neuropsin generation, etc.) using common, daily-use devices.

In one embodiment, the present invention involves a system for delivering light therapy for entraining brainwaves. In one embodiment, the system comprises: a pulsed light source configured for emitting pulsed light oscillating between first and second states; (a) wherein the first and second states comprise at least one of (1) different intensities of the same light color, (2) different light colors, (3) different intensities of different light colors; and (4) metamers; (b) wherein the pulsed light source is configured to minimize a user's visual detection during oscillation between the first and second states, wherein minimizing visual detection comprises at least one of (1) combining pulsed light with continuous light, (2) gaze-based light therapy, (3) activity-dependent light therapy, (4) metamer use, (5) duty cycle moderation, (6) waveform variation, and combinations thereof.

In one embodiment, the present invention involves a method of using a system as described above.

In one embodiment, the present invention involves a method of delivering BLT. In one embodiment, the method comprises delivering pulsed light to a user, the pulsed light oscillating between first and second states; (a) wherein the first and second states comprise at least one of (1) different intensities of the same light color, (2) different light colors, (3) different intensities of different light colors; and (4) metamers; (b) wherein the pulsed light source is configured to minimize a user's visual detection during oscillation between the first and second states, wherein minimizing visual detection comprises at least one of (1) combining pulsed light with continuous light, (2) gaze-based light therapy, (3) activity-dependent light therapy, (4) metamer use, (5) duty cycle moderation, (6) waveform variation, and combinations thereof.

### FIGURES

FIG. 1 shows one embodiment of the system of the present invention.

FIG. 2 shows a spectrogram for constant white+flickering red (top) and constant white only (bottom) conditions at Oz location.

FIG. 3 shows 40 Hz power registered from the electrodes on the z-locations Short- and long-term impact of circadian light interventions on older adults with AD/ADRD.

FIG. 4 shows one embodiment of a monitor embodiment of the present invention.

FIG. 5 shows one embodiment of a floor lamp embodiment of the present invention.

FIG. 6 shows one embodiment of a gaze-based brainwave light therapy.

### DETAILED DESCRIPTION

One aspect of the present invention is a system for entraining brainwaves in a user's brain using pulsed or flickering light (referred to herein as brainwave light therapy (BLT)). Although the entrainment of gamma brainwaves is considered specifically in this disclosure, it should be understood, the present invention is not limited to entraining gamma waves, and covers pulsing light to entrain any desired brainwave, which may or may not include gamma waves. For example, in one embodiment, the pulsed light is configured to entrain one or more of the following brainwaves: gamma (associated with problem-solving/concentration), beta (associated with busy, active mind), alpha (associated with reflective/restful thought), theta (associated with drowsiness), and delta (associated with sleep/dreaming). For example, in one embodiment, a pulsed light having a frequency of between 40 and 200 Hz in integer multiples of 20 Hz is used to entrain gamma waves in the person.

The pulsed light may take on different embodiments within the scope of the invention. For example, in one embodiment, the pulsed light has oscillating states, wherein the oscillating states comprise at least a first state and a second state. In one embodiment, the first and second states are (1) different intensities of the same light color, (2) different light colors, (3) different intensities of different light colors; and (4) metamers—i.e., light with perceived matching of colors but different (nonmatching) spectral power distributions (SPDs).

For example, in one embodiment, the intensity of one color is oscillated between a relatively high intensity (first state) and a low intensity (second state) at 40 Hz. The difference in intensities may be, for example, the relative difference in brightness between the two states, or, in a more extreme embodiment, the light may be “on” in the first state, and may be “off” in the second state.

Light has luminance and chrominance components, among others. Although the tendency is to maintain chrominance, and vary luminance in pulsed light, both may be varied independently. For example, luminance may be held constant, and the flicker signal may be delivered by changing chrominance. For example, in one embodiment, rather than alternating the intensity of the light, lights of different color can be oscillated to effect a 40 Hz oscillation cycle. For example, in one embodiment, a perceptively blue light may be used in the first state, and a perceptively red light may be used in the second state. In another example, a white light may be used as a baseline condition and monochromatic or colored light may be used to add a pulsed light component.

In yet another embodiment, both the color and the intensity of the light may be oscillated. For example, continuing the example above, in one embodiment, the blue light (first state) may have a lower intensity than the red light (second state).

In the metamer embodiment, the first and second states have the same perceived colors and intensities, but they have different (nonmatching) spectral power distributions (SPDs). This embodiment is described in greater detail below.

### I. Minimally Intrusive BLT

As mentioned above, Applicant understands that if BLT is to be successful, it must be innocuous/inconspicuous. Conversely, a 40 Hz, 50% duty cycle, square wave light therapy would be irritating and uncomfortable, and hence, the likelihood of long term compliance with the BLT would be remote. Accordingly, Applicant discloses herein various approaches for minimizing the obtrusiveness of BLT, including, for example, just to name a few, (1) combining light therapy with continuous light; (2) gaze-based light therapy; (3) activity-dependent light therapy; (4) metamer use; (5) duty cycle moderation; (6) waveform variation; and combinations thereof.

(1) Combining with Continuous Light

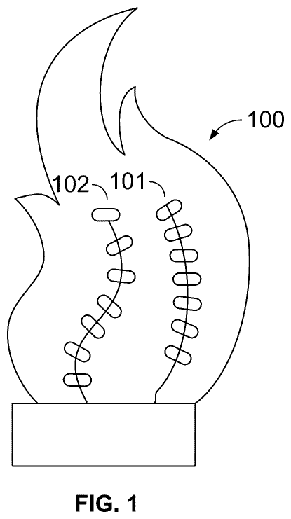

In one embodiment, continuous light is combined with BLT to obscure, or otherwise diminish, the visual impact of the pulsed light on the user. In one embodiment, the continuous light is white light. Nevertheless, it should be understood that those of skill in the art in light of this disclosure may substitute nonwhite light as a continuous light to mute the effect of the pulsed light on the user. For example, referring to FIG. 1, one embodiment of a system 100 for delivering light therapy is shown. In this embodiment, the system 100 comprises: a first light source 101 for emitting continuous light; and a second light source 102 for emitting pulsed light having a frequency of between 40 and 200 Hz in integer multiples of 20 Hz.

### (2) Gaze-Based Light Therapy

Pulsed light is perceived differently depending upon where it falls in the visual field. There is also evidence of a relationship between the strength of brainwave entrainment and the visual field location and the portion of field occupied. That is, Applicant has found a significant relationship between the region of the visual field stimulated and the sensitivity of the entrainment response. Similarly, Applicant has found a relationship between the region of the visual field stimulated and perceivability and acceptability of the stimulus exists.

The efficiency of gamma entrainment is determined by several factors, a significant one being gaze angle. Light induced gamma activation is determined by the central vision, which makes digital displays, such as televisions and computer screens, among the most effective means to deliver gamma stimulation. Additionally, gamma activity is increased when user's stare directly at the 40 Hz flickering light source. For example, when flickering stimuli with visual angles of 2⁰ and 10⁰ were positioned on users' central vision, gamma activity was significantly enhanced. However, when the sources with the identical visual fields were placed on the periphery (i.e., 25 cm away from the line of vision), no detectable 40 Hz activity was observed. Thus, displays such as televisions and computer screens may be the most effective means of administering 40 Hz stimulation because viewers stare directly at these devices during normal use.

In one embodiment, the present invention relates to a system that uses eye tracking or other methods to determine the location of gaze. The system then delivers 40 Hz stimulation from a region of the screen selected to maximize the benefit and acceptability and minimize side effects.

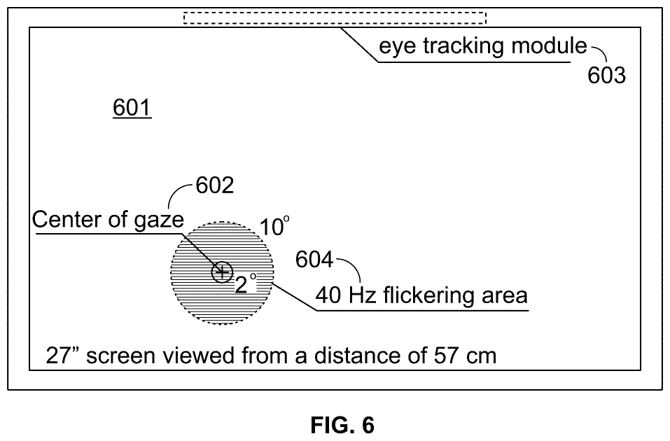

For example, referring to FIG. 6, one embodiment of a gaze-based brainwave light therapy system is shown. The system tracks the user's eyes using an eye tracking module 603 to determine the user's focus 602 on-screen 601. In this embodiment, an BLT area 604 around the focus 602 is controlled to emit pulsed light. In this embodiment, the user focus is determined to be 2° based on a 27″ screen viewed from a distance of 57 cm, and the BLT area 604 is determined to be 10°. It should be understood that these areas will change based on the screen size and user's distance from the screen. Also, it should be understood, that the BLT area may be optimized, and thus, may be greater or less than 10°. In one embodiment, only the BLT area 604 around the focus 602 is pulsed (i.e., the focus 602 is not pulsed). Alternatively, in one embodiment, the BLT area including the focus 602 is pulsed.

Still other embodiments of a gaze-based BLT system will be obvious to those of skill in the art in light of this disclosure. For example, rather than pulsing the area around the user's focus, it may be more effective to pulse the area of the screen outside of the user's focus. In other words, rather than pulsing BLT area as illustrated above, it may be preferable to pulse the area of the screen outside of the BLT area. Such embodiment may be preferred if it is determined that BLT is more effective and/or less obtrusive when delivered to the user's peripheral vision. Likewise, it may be preferable to deliver BLT preferentially above the user's focus rather than on either side of the focus due to the human eye's particular sensitivity to light above the horizon. Again, this becomes a question of optimization—i.e., balancing intrusiveness with effectiveness—which would be understood by one of skill the art in light of this disclosure.

In one embodiment, the system is configured to track independently multiple users and provide them with targeted BLT. In one embodiment, the system may opt to suspend BLT if there is no way to avoid excessive irritation, or the system may provide an alternative invisible or minimally visible BLT (e.g., infrared light).

### (3) Activity-Dependent Light Therapy

Applicant has identified a task dependence for the acceptability of the BLT, meaning that certain tasks are more likely to be disturbed by BLT stimulation. For example, 40 Hz stimulation is more noticeable while composing an email than when watching a video. Accordingly, BLT may be delivered during certain tasks in which the user is less sensitive to flickering light. In one embodiment, the system is configured to identify the active window on a computer and the task being performed. Depending upon the task, the system delivers BLT in a manner that maximizes the benefit and acceptability, and minimizes side effects as disclosed herein. In one embodiment, if the task being performed does not lend itself to BLT, the system is configured to deliver an invisible or minimally visible BLT (e.g., infrared light) and/or to deliver the BLT from a source outside the field of view (e.g., in the bezel).

### (4) Metameric Pulsed Light

Applicant submits that pulsed light between two metameric light sources may allow for the delivery of an invisible entrainment signal. Metamers are color stimuli that have different spectral radiant power distributions but are perceived as identical for a given observer. Metamers are, by definition, visually indistinguishable to a user. However, there are non-image-forming sensors in the human eye that can receive different signals despite the inputs being metamers. This can be used to deliver an invisible flicker signal. Accordingly, in one embodiment, the first and second states are metamers.

For example, referring to FIG. 1, in this embodiment, the second light source oscillates between first and second metamers. For example, the first metamer may be white light having a relatively high melanopic to photopic (m/p) ratio, and the second metamer would have the same perceptively white light, although with a relatively low m/p ratio. The m/p ratio is well known, and is the ratio of the melanopic (ipRGC) potential to the light source's ability to produce light for daytime detail vision (photopic vision). Generally, a low m/p ratio is desirable for low EML light and a high m/p ratio is desirable for high EML light. Still other configurations for effecting BLT using metamers will be obvious to those of skill the art in light of this disclosure.

Light sources for effecting first and second metamer states are commercially available and include, for example, Vigor, Dynamic Vivid, and a 2-channel backlit LCD available from Korrus, Inc.

### (5) Duty Cycle

Another approach for moderating the intrusiveness of BLT is to vary the duty cycle. The duty cycle may be varied in different ways, including, for example, (1) the relative duration of the first and second states; (2) depth of the oscillation between first and second states; and (3) transition shape between the first and second states.

The duty cycle of the first and second states may vary. The duty cycle is the duration of the first state over the total duration of the first and second states. In one embodiment, the duty cycle is greater than 0% and less than 100%. In one embodiment, the duty cycle is no less than 25% and no greater than 75%. In one particular embodiment, the duty cycle is about 50%. It will be obvious to one of skill in the art in light of this disclosure to tune the duty cycle the balance effectiveness and intrusiveness.

In addition to the relative time the first and second states are “on,” another important parameter is the difference between the two states or “cycle depth.” That is, if the first state has a color or intensity of 100%, for example, the second state may have a color or intensity greater than zero or “off,” but something less than 100% on—e.g. 80 or 70%. In this way, the difference between the first and second states is not on-off, but rather is a relative change—e.g. 100 to 80% or 100 to 70%. Applicant expects that going from full on to full off (i.e. 100% to 0%) may be more effective in training brainwaves, but its effectiveness is outweighed by the annoyance of such a harsh difference between the first and second states, thus militating in favor of a more innocuous BLT with a shallower cycle depth. Accordingly, in one embodiment, the ratio of intensity between the first and second state will one to something less than one—e.g., 1:9, or, 1:8, or, 1:0.75, or, 1:0.7, or 1:0.65, or 1:0.6, or, 1:0.55, or 1:0.5. Still other ratios exist within the scope of the invention.

### (6) Waveform

The form of the oscillation may also vary. For example, in one embodiment, the oscillation between the two states has the form of a square wave, sinusoidal wave, or triangular wave. In other words, the transition from the first state to the second state may be abrupt, e.g., square waveform, or more tapered, e.g., a sinusoidal wave or triangular waveform. Those of skill in the art in light of this disclosure will be able to determine without undue experimentation the optimal waveform. For example, depending upon the perceptible difference between the first and second states, it may be preferable to extend the transition between the first and second states. This may be the case, for example, if the difference between the first and second states is particularly apparent to the naked eye. On the other hand, if the difference between the first and second states is not particularly noticeable to the naked eye, it may be preferable to abruptly switch between the first state and the second state, and, thus, a square wave may be preferred. For example, if the second light source emits metamers in the first and second states, then an abrupt transition between the states may be fine because the difference between metamers is not discernable to the naked eye. Furthermore, even if the difference between the first and second states of the pulsed light is noticeable, it may be preferable to use a square wave because a more abrupt change between the first and second states may be more effective in entraining gamma waves. One of skill the art in light of this disclosure will be able to configure the oscillation transition between the first and second states to optimize brainwave entrainment and user comfort.

In one embodiment, the waveform may be a base frequency but with added harmonics to soften or mellow the light. This may be described in terms of a Fourier series. Fourier series allow for the representation of arbitrary, periodic waveforms through a combination of sine and cosine waves of differing amplitudes, frequencies, and phases. A waveform with a period corresponding to a frequency will have a dominant component at that frequency. Additional components, for example harmonics, can change the character of the waveform to make it more tolerable. By way of a comparison, consider a piano note: Middle C has a dominant frequency of approximately 262 Hz. However, it sounds different to a pure sinewave at that same frequency. While subjective, most people will agree that middle C on a piano has a nicer sound than a pure sine wave with the same frequency. It is this notion of combining the basis “note” with harmonics to produce a more tolerable stimulation that is described here. (See, e.g., https://www.projectrhea.org/rhea/index.php/Fourier_analysis_in_Music, which shows the weights of different harmonics for a variety of instruments.)

### Square Wave:

$$\begin{matrix}x ( t ) = \frac{4}{π} {\sum }_{k = 1}^{∞} \frac{sin ( 2 π ( 2 k - 1 ) ft )}{2 k - 1} \\ = \frac{4}{π} ( sin ( ω t ) + \frac{1}{3} sin ( 3 ω t ) + \frac{1}{5} sin ( 5 ωt ) + … ) , where ω = 2 π f .\end{matrix}$$

### Triangle:

$${x}_{triangle} ( t ) = \frac{8}{{π}^{2}} {\sum }_{i = 0}^{N - 1} {( - 1 )}^{i} {n}^{- 2} sin ( 2 π {f}_{0} nt )$$

The power of the pulsed light waveform therefore may be described as the ratio root-mean-square of the target frequency component to the root-mean-square of the total waveform. For example, for a square wave the ratio may be 0.71:1, and for a sine wave the ratio may be 1.00:1; and for a triangle wave the ratio may be: 0.99:1.

### II. Combining Light Therapies

To promote user compliance with BLT over the long term, Applicant discloses herein approaches for synergistically combining BLT with other light therapies. Such synergistic combinations include combining BLT with (1) circadian cycle light therapy, (2) non-circadian light stimulation, and (3) neuropsin stimulation.

### (1) Circadian Cycle Light Therapy

Aside from degradation of gamma waves in the brain, the destruction of circadian rhythms may also contribute to AD. In approximately 45% of Alzheimer's patients, circadian rhythm problems and sleep disruptions have been recorded, where a bidirectional association between sleep and Alzheimer's disease is suggested, with sleep abnormalities serving as either a marker for AD pathology or a mechanism mediating increased risk of the disease. Therefore, in one embodiment, in addition to stimulating gamma brainwaves, regulating the circadian cycle of AD patients may also be useful. (It should be understood, however, that the gamma entrainment function and the circadian rhythm moderation function of the present system may be used to treat others, outside of AD patients.)

Applicant recognizes that it is possible to alter both circadian rhythms and brainwave activity using light entering the eye. Further, it is possible to do both at the same time without one compromising the other. As a simple example, flickering blue light maybe used during the portion of the circadian cycle when circadian input is desirable (‘day’) and flickering red light may be used when circadian input should be avoided (‘night’).

In one embodiment, the BTL and circadian cycle light therapy are coordinated. For example, in one embodiment, during “day,” a high circadian stimulation light may be used while the BLT may target gamma waves. Such an embodiment would have the effect of stimulating the patient. In another embodiment, during “night,” a low circadian stimulation light may be used, while the BLT targets theta waves. Such an embodiment would have the effect of relaxing the patient. Still other combinations of circadian stimulation light and BLT will be obvious to those of skill he art in light of this disclosure.

Accordingly, in one embodiment, the system of the present invention is designed to support both circadian rhythms and BLT. For entrainment purposes, in one embodiment, it features a steady state light combined with a pulsing light. The combination of steady light with pulsing light is intended to improve the usability of the system. As mentioned above, a flickering light can be aversive, but with the addition of a continuously emitting light source, the oscillating effect of a light can be muted. It is important that any light therapy system be non-obtrusive. Preferably the light should even induce patients to look at it. AD patients can be difficult to treat because they do not understand what is happening. It is therefore important that any intervention be sufficiently acceptable that they do not try to avoid or stop it. This is a common problem with goggle-type interventions.

To support circadian rhythms, in one embodiment, the continuous light varies between a cool, high m/p source and a warm, low m/p source and the colored component is a high m/p source during the day (˜485 nm) and a low m/p source at night (600-700 nm). In one embodiment, the system is embodied in a self-contained device such as a display, desktop lamp, or tabletop sculpture.

The color of the pulsed light may vary and may be selected to optimize not only gamma wave entrainment, but also moderation of circadian rhythms. In one embodiment, the color is a monochrome light. In one embodiment, the monochrome light is a perceptually non-white color. In one embodiment, the perceptually non-white color is a color that activates only a portion of light receptors of a human eye. In one embodiment, the perceptually non-white color is one of red or violet. In one embodiment, the monochrome light stimulates only a portion of light receptors of a human eye. In one embodiment, the monochrome light stimulates non-visual photoreceptors in the human eye.

Referring to FIG. 1, in one embodiment, at least one of the first light source or the second light source has a relatively high EML state for emitting a high EML light, and a relatively low EML state for emitting a low EML light. In a more particular embodiment, the low EML light has a low EML spectral power distribution (SPD), wherein the low EML SPD has a low EML total power between 380 nm to 790 nm, and a low EML blue power between 440 nm to 490 nm, wherein the low EML blue power is less than 2% of the low EML total power. In one embodiment, the high EML light has a high EML spectral power distribution (SPD), wherein the high EML SPD has a high EML total power between 380 nm to 790 nm, and a high EML blue power between 440 nm to 490 nm, wherein the high EML blue power is at least 10% of the high EML total power. In yet another embodiment, the relatively high EML state and the relatively low EML state have varying m/p ratios. The m/p ratio is well known, and is the ratio of the melanopic (ipRGC) potential to the light source's ability to produce light for daytime detail vision (photopic vision). Generally, a low m/p ratio is desirable for low EML light and a high m/p ratio is desirable for high EML light. Accordingly, in one embodiment, the m/p ratio for the relatively low EML state light is less than 1.5, 1.3, 1.1, 1.0, 0.9, or 0.8, and the m/p ratio for the relatively high EML state light is greater than 0.9, 1.0, 1.1, 1.2, 1.5, 2.0, or 2.5.

It should be understood that either the first light source or the second light source may provide emitted light for moderating circadian rhythms. In one embodiment, the first light source has a high EML state and the low EML state. In another embodiment, the second light source has the high EML state and the low EML state. In yet another embodiment, both light sources have high and low EML states. In yet another embodiment, the first light source has a high EML state, and the second light source has a low EML state. In still another embodiment, the first light source has the low EML state, and the second light source has the high EML state.

In one embodiment, the circadian light therapy is provided by a display. For example, a two-channel backlit LCD display may be set to flicker between high and low EML modes at 40 Hz to induce gamma waves and enhance cognitive performance. In another example, illumination and a 2-channel backlit TV may flicker between high and low EML at 4-8 Hz to promote theta waves and relaxation.

### (2) Non-Circadian Stimulation

Aside from circadian stimulation, the BLT of the present invention be combined with other non-circadian stimulation light therapies. For example, in one embodiment, the user is exposed to barely perceptible pulsing red light which has been found to energize humans without interfering with their circadian cycles. For example, often people feel lethargic after lunch and require stimulation without jumpstarting their circadian cycle. In such a case, flashing red or infrared light has been shown to provide stimulation. Applicant submits that such non-circadian stimulation combined with BLT targeting gamma waves provide a synergistic solution to stimulating users and minimizing the after-lunch dip.

### (3) Stimulation of Neuropsin

Neuropsin is a protein that contributes to establishing circadian rhythms in a mammal's eye. Specifically, the behavioral circadian rhythms of mammals are synchronized to light/dark cycles through rods, cones, and melanopsin-expressing, intrinsically photosensitive ganglion cells in the retina. However, the molecular circadian rhythms in the mammalian retina are synchronized to light/dark signals using neuropsin. Additionally, the circadian clocks in the cornea are also photoentrained using neuropsin.

It has been found that shorter wavelength light (e.g. violet) stimulates neuropsin. Therefore, to regulate a person's circadian rhythm, it may be preferable to subject the user not only to light containing various levels of blue/cyan light, but also to violet light for neuropsin stimulation. In other words, while the body's circadian cycle is regulated by blue/cyan light, the eye has its own circadian cycle which is regulated by violet light. Accordingly, in one embodiment, a holistic circadian cycle light therapy involves dosing the user with both blue/cyan light and violet light. Applicant also suspects that dosing with violet light may help prevent myopia.

Accordingly, in one embodiment, the BLT of the present invention is combined with violet light to stimulate neuropsin and regulate circadian rhythm of a person's eye.

III. Integration with Daily-Use Items

Applicant recognizes that to ensure long-term compliance with BLT (plus any other light therapy it may be combined with), the device that provides the light should be something that the user willingly engages with daily. Accordingly, in one embodiment, the BLT is embodied in a display (computer or TV), room lighting, or decorative accessory.

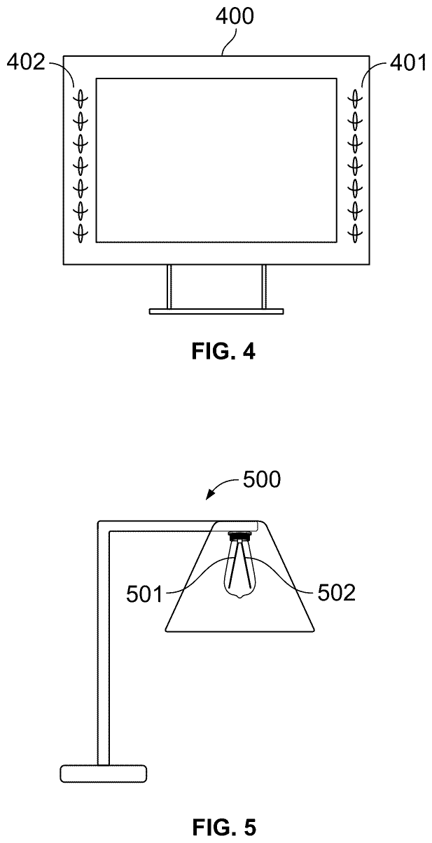

For example, referring to FIG. 4, in one embodiment, a panel 400 comprises a first light source 401 and a second light source 402. In one embodiment, the panel is a vertical panel or a table-top panel. In one embodiment, the panel is a display panel for displaying digital content. Referring to FIG. 1, an alternative embodiment of the system 100 has a housing comprising a sculpture. In one embodiment, the sculpture is a table-top sculpture. In a particular embodiment, the table-top sculpture is an orb or a statue. In such an embodiment, light may emit from the sculpture in all directions. Referring to FIG. 5, an alternative embodiment of the system is shown comprises a lamp 500 having first and second light sources 501, 501. In one embodiment, the lamp is a table-top lamp.

Considering displays in particular, in one embodiment, a backlit LCD has a backlight that is modulated at 40 Hz to deliver stimulation. In another embodiment, the LCD has one or more pixels that are modulated to deliver stimulation. In a miniLED display, one or more zones may be modulated at 40 Hz to deliver stimulation. In an emissive display, one or more pixels may be modulated at 40 Hz to deliver stimulation. Still other embodiments will be obvious to those of skill in the art in light of this disclosure.

### Examples

Applicant previously conducted a feasibility study of 40 Hz flickering light to enhance gamma oscillations, improve working memory performance, and reduce subjective sleepiness in healthy young adults using red light, to which the human circadian system is less sensitive. The results of his work were published in the Journal of Alzheimer's Disease in 2020. In the study, custom-built light masks provided either a 40 Hz FL intervention at T2 and T3 or a dark control condition (no-flicker) at T1 and T4. His results from 9 healthy participants demonstrated that the FL intervention induced a significant increase in 40 Hz power, specifically at the parietal (Pz) and occipital (Oz) lobes (FIG. 1) using electroencephalography (EEG). Increased 40 Hz power was associated with decreased subjective sleepiness. The intervention had no effect on working memory, possibly because the subjects were well-rested, healthy young adults.

Applicant has also shown that circadian appropriate light improved measures of circadian entrainment, sleep efficiency, and reduced depression and agitation in patients with ADRD.

Initial tests of the BLT prototype with limited functionality. Previously, Applicant demonstrated the efficacy of light mask flickering at 40 Hz for gamma entrainment. Using light masks, however, is not a suitable medium for delivering light to AD patients. To this end, more recently, Applicant conceptualized and designed a preliminary BLT prototype with limited functions to provide either 40 Hz flickering red light in combination with constant white light or only constant white light.

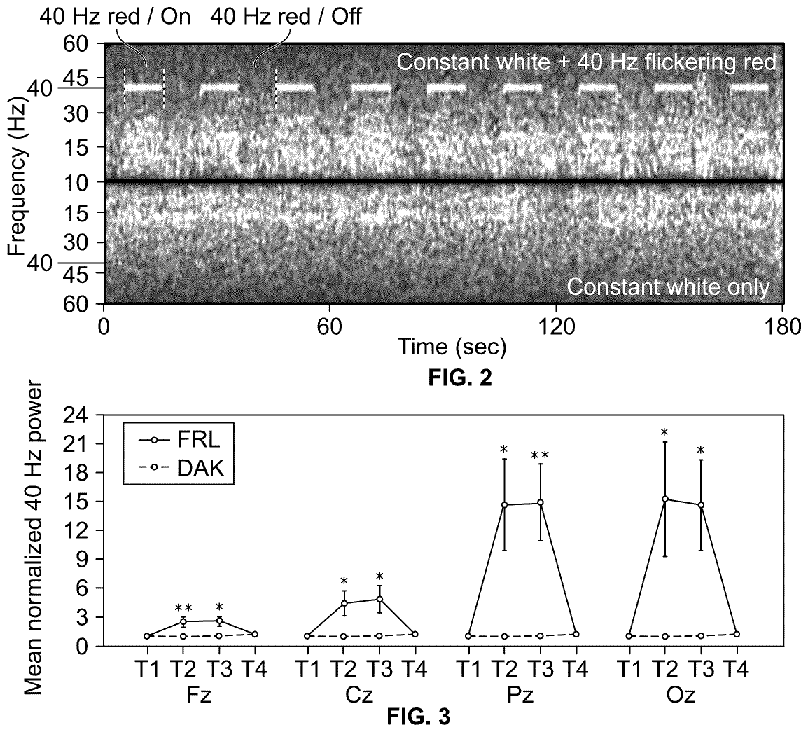

The prototype's gamma entrainment was assessed in two lighting situations on a 45-year-old participant. During the 180 seconds (sec) of EEG recordings, the participant was exposed to either the combined condition (constant white+40 Hz flickering red) or just constant white light condition. In the combined condition, the flickering red light was on and off for 10 sec (as marked on FIG. 2-top row) while the constant white was on during the entire session. In line with our hypothesis, the BLT prototype entrained gamma activity at the Oz location during red flickering stimulation presented with constant white (FIG. 2, top row). When constant light presented alone, however, the gamma entrainment was not observed at the 40 Hz frequency band (FIG. 2, bottom row).

These and other advantages maybe realized in accordance with the specific embodiments described as well as other variations. It is to be understood that the above description is intended to be illustrative, and not restrictive. Many other embodiments and modifications within the spirit and scope of the claims will be apparent to those of skill in the art upon reviewing the above description. The scope of the invention should, therefore, be determined with reference to the appended claims, along with the full scope of equivalents to which such claims are entitled.

What is claimed is:

1. A system for delivering light therapy for entraining brainwaves, the system comprising:
a pulsed light source configured for emitting pulsed light oscillating between first and second states;
wherein said first and second states comprise at least one of (1) different intensities of the same light color, (2) different light colors, (3) different intensities of different light colors, and (4) metamers; and
wherein said pulsed light source is configured to minimize a user's visual detection during oscillation between said first and second states, wherein minimizing visual detection comprises at least one of (1) combining pulsed light with continuous light, (2) gaze-based light therapy, (3) activity-dependent light therapy, (4) metamer use, (5) duty cycle moderation, (6) waveform variation; and combinations thereof.

2. The system of claim 1, wherein said pulsed light is configured to entrain one or more of the following brainwaves: gamma (associated with problem-solving/concentration), beta (associated with busy, active mind), alpha (associated with reflective/restful thought), theta (associated with drowsiness), or delta (associated with sleep/dreaming).

3. The system of claim 1, further comprising functionality for delivering an additional light therapy comprising at least one of (1) circadian cycle light therapy, (2) non-circadian light stimulation, or (3) neuropsin stimulation.

4. The system of claim 1, wherein said pulsed light has an oscillation frequency of between 40 and 200 Hz in integer multiples of 20 Hz.

5. The system of claim 1, further comprising a continuous light source configured for emitting continuous light.

6. The system of claim 5, wherein said continuous light is white light.

7. The system of claim 6, wherein said white light is modulated to moderate circadian cycles.

8. The system of claim 7, wherein said while light has a relatively high EML state for emitting a high EML light, and a relatively low EML state for emitting a low EML light.

9. The system of claim 8, wherein in the low EML light has a low EML spectral power distribution (SPD), wherein the low EML SPD has a low EML total power between 380 nm to 780 nm, and a low EML blue power between 440 nm to 490 nm, wherein the low EML blue power is less than 2% of the low EML total power.

10. The system of claim 9, wherein the high EML light has a high EML spectral power distribution (SPD), wherein the high EML SPD has a high EML total power between 380 nm to 780 nm, and a high EML blue power between 440 nm to 490 nm, wherein the high EML blue power is at least 10% of the high EML total power.

11. The system of claim 5, wherein the continuous light source has a high EML state and a low EML state.

12. The system of claim 1, wherein said gaze-based light therapy comprises determining focus area of user gaze, and delivering said pulsed light from a region of the screen based on said focus area.

13. The system of claim 1, wherein said activity-dependent light therapy comprises identifying an active window on a computer and task being performed, and, based on said task, delivering pulse light in a manner that maximizes the benefit and acceptability, and minimizes side effects as disclosed herein.

14. The system of claim 1, wherein said first and second states define a duty cycle.

15. The system of claim 14, wherein said duty cycle comprises a square wave of a first duration of the first state over the total duration of the first and second states, wherein the duty cycle is greater than 0% and less than 100%.

16. The system of claim 15, wherein the duty cycle is no less than 25% and no greater than 75%.

17. The system of claim 1, further comprising a housing in which said pulse light source is contained.

18. The system of claim 17, wherein housing comprises a panel.

19. The system of claim 18, wherein the panel is a vertical panel or a table-top panel.

20. The system of claim 18, wherein the panel is a display panel for displaying digital content.

21. The system of claim 17, wherein the housing comprises a sculpture.

22. The system of claim 14, said pulsed light source is within a lamp.

23. The system of claim 1, wherein said pulsed light has a waveform expressed as a Fourier series in which at least 5%, or 10%, or 15%, or 20%, or 25%, or 30%, or 35%, or 40%, or 50%, or 60%, or 70% of the power of the waveform is at the desired frequency for BLT (e.g., 40 Hz for gamma waves).

24. A system for delivering light therapy, the system comprising:
a first light source for emitting continuous light; and
a second light source for emitting pulsed light having a frequency of between 40 and 200 Hz in integer multiples of 20 Hz.

25. The system of claim 24, wherein the pulsed light has oscillating states, wherein the oscillating states comprise at least a first state and a second state wherein the first and second states are (1) different intensities of the same light color, (2) different light colors, (3) metamers, or (4) different intensities of different light colors.

26. The system of claim 25, wherein the first and second states are (1) different intensities of the same light color, (2) different light colors, (3) metamers, or (4) different intensities of different light colors.

27. The system of claim 26, wherein the transition between the first state and the second state is in the form of at least one of a square wave, sinusoidal wave, or triangular wave.

28. The system of claim 27, wherein the square wave as a duty cycle of a first duration of the first state over the total duration of the first and second states, wherein the duty cycle is greater than 0% and less than 100%.

29. The system of claim 28, wherein the duty cycle is no less than 25% and no greater than 75%.

30. The system of claim 25, wherein the first and second states are different intensities of the same light color.

31. The system of claim 30, wherein the first state is on and the second state is off.

32. The system of claim 31, wherein the same color light is a perceptually non-white color light that activates only a portion of light receptors of a human eye.

33. The system of claim 32, wherein the monochrome light is a perceptually non-white color.

34. The system of claim 33, wherein the perceptually non-white color is a color that activates only a portion of light receptors of a human eye.
